(19) United States
(12) Patent Application Publication
(10) Pub. No.: US 2010/0249619 A1
(43) Pub. Date: Sep. 30, 2010

(54) PHYSICAL FITNESS LEVEL DETERMINING APPARATUS, METHOD AND MOBILE TERMINAL APPARATUS

(75) Inventor: Koichiro KASAMA, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 12/726,614

(22) Filed: Mar. 18, 2010

(30) Foreign Application Priority Data
Mar. 30, 2009 (JP) 2009-083014

(51) Int. Cl. A61B 5/024

(52) U.S. Cl. 600/502

(57) ABSTRACT

An apparatus for determining physical fitness levels, includes: a pulse measuring unit which measures a pulse rate of a user at rest; a measurement processor which acquires a pulse rate ratio of the pulse rate relative to a maximum pulse rate which is determined by an actual age of the user, estimates an oxygen uptake ratio on the basis of the pulse rate ratio and the actual age, and calculates a maximum oxygen uptake from an oxygen uptake at rest by using the oxygen uptake ratio; a conversion table which associates a maximum oxygen uptake with a physical fitness level; and a determiner which determines the physical fitness level in accordance with the calculated maximum oxygen uptake and the conversion table.

### CROSS-REFERENCE TO RELATED APPLICATION

This application is based upon and claims the benefit of priority of the prior Japanese Patent Application No. 2009-083014, filed on Mar. 30, 2009, the entire contents of which are incorporated herein by reference.

### FIELD

A certain aspect of the embodiments discussed herein relates to a technology to determine the physical fitness level or the physical age of a person by using the pulse and the oxygen uptake.

### BACKGROUND

It is said that the physical fitness levels and the physical ages of persons depend on, for example, the strengths of their cardiopulmonary functions. Persons who continually exercise generally tend to have higher physical fitness levels and younger physical ages. Persons having younger physical ages or higher physical fitness levels have lower heart rates during exercise and the heart rates quickly return to those at rest after the exercise stress is released.

In determination of the physical ages or the physical fitness levels, the heart rates, etc. of persons are measured in a state in which the persons do exercise with stress actually applied thereto.

As for the physical fitness levels and the physical ages, Japanese Laid-open Patent Publication No. 6-105830 discloses calculation of maximum heart rate and evaluation of physical fitness on the basis of an evaluation table using oxygen uptake, Japanese Laid-open Patent Publication No. 6-245914 discloses calculation of estimation values of pulse rate, exercise stress level, etc. by using a multivariate model equation, and Japanese Laid-open Patent Publication No. 63-132636 discloses calculation of physical fitness level or physical age by using a regression equation indicating the correlation among power, oxygen uptake, pulse rate, and the like.

[Patent document 1] Japanese Laid-open Patent Publication No. 6-105830

[Patent document 2] Japanese Laid-open Patent Publication No. 6-245914

[Patent document 2] Japanese Laid-open Patent Publication No. 63-132636

In the determination of the physical ages or the physical fitness levels, for example, variations in the heart rates of persons are measured while the persons are actually doing exercise to measure more precise physical tolerances. In such measurement, it is necessary for the persons to continually exercise for certain times in order to achieve stable results. When the physical ages or the physical fitness levels of general users are measured to indicate references for daily health care, it is difficult to realize the critical load by exercise.

Furthermore, physical feature data indicating body weights, body heights, etc. is considered as personal information, in addition to, for example, age information, in detailed physical fitness measurement. The measurement of the physical feature data prevents the physical ages from being easily calculated.

Japanese Laid-open Patent Publication Nos. 6-105830, 6-245914, and 63-132636 do not disclose and suggest the above demands, problems, and configuration to resolve the problems.

### SUMMARY

According to an aspect of an embodiment, an apparatus for determining physical fitness levels, includes: a pulse measuring unit which measures a pulse rate of a user at rest; a measurement processor which acquires a pulse rate ratio of the pulse rate relative to a maximum pulse Tate which is determined by an actual age of the user, estimates an oxygen uptake ratio on the basis of the pulse rate ratio and the actual age, and calculates a maximum oxygen uptake from an oxygen uptake at rest by using the oxygen uptake ratio; a conversion table which associates a maximum oxygen uptake with a physical fitness level; and a determiner which determines the physical fitness level in accordance with the calculated maximum oxygen uptake and the conversion table. The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating an example of the functional configuration of a physical fitness determining apparatus according to a first embodiment of the present invention;

FIG. 2 is a block diagram illustrating an example of the hardware configuration of the physical fitness determining apparatus in FIG. 1;

FIG. 3 is a block diagram illustrating an example of the configuration of a memory in the physical fitness determining apparatus in FIG. 1;

FIG. 4 illustrates an exemplary state of measurement of a pulse rate by a camera-type pulse measuring unit;

FIG. 5 is a flowchart illustrating an example of a process of measuring the pulse rate;

FIG. 6 is a flowchart illustrating an example of a process of calculating the pulse rate;

FIG. 7 is a flowchart illustrating an example of a process of determining physical fitness;

FIG. 8 illustrates an example of a coefficient table;

FIG. 9 is a flowchart illustrating an example of a process of determining a physical fitness level;

FIG. 10 illustrates an example of how to determine the physical fitness level;

FIG. 11 is a flowchart illustrating an example of a process of determining a physical age;

FIG. 12 illustrates an example of how to determine the physical age;

FIG. 13 is a flowchart illustrating an example of a process in a display processor according to a second embodiment of the present invention;

FIG. 14 illustrates an example of how to display the physical fitness level and an advice;

FIG. 15 illustrates an example of how to display the physical age and an advice;

FIG. 16 illustrates an example of displayed guidance in pulse measurement;

FIG. 17 illustrates an example of displayed guidance;

FIG. 18 is a block diagram showing an example of the hardware configuration of a portable terminal apparatus according to a third embodiment of the present invention;

FIG. 19 illustrates an example of the external structure of the portable terminal apparatus in an open state;

FIG. 20 illustrates an example of the external structure of the portable terminal apparatus in a closed state;

FIG. 21 illustrates an example of the structure of an ear-clip-type pulse measuring unit according to a fourth embodiment of the present invention;

FIG. 22 illustrates an example of how to wear the ear-clip-type pulse measuring unit;

FIG. 23 is a flowchart illustrating an example of a process of calculating the pulse rate in the ear-clip-type pulse measuring unit;

FIG. 24 illustrates a PDA according to another embodiment of the present invention; and

FIG. 25 illustrates a PC according to another embodiment of the present invention.

### DESCRIPTION OF EMBODIMENTS

### First Embodiment

A physical fitness determining apparatus, a physical fitness determining method, a physical fitness determining program, and a portable terminal apparatus according to a first embodiment of the present invention calculate a maximum oxygen uptake by using a pulse rate that is acquired to determine the physical age or the physical fitness level from the calculated maximum oxygen uptake by conversion means.

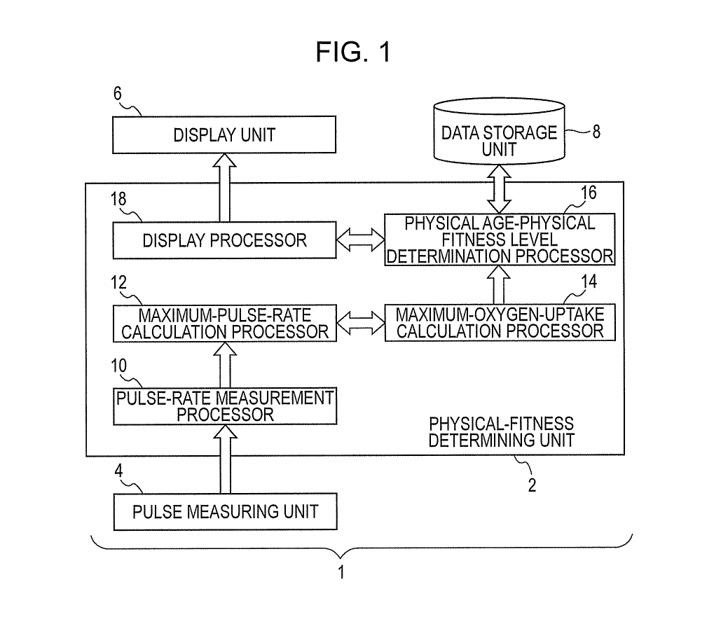

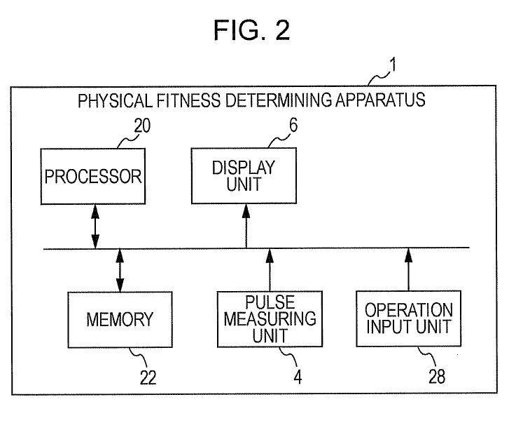

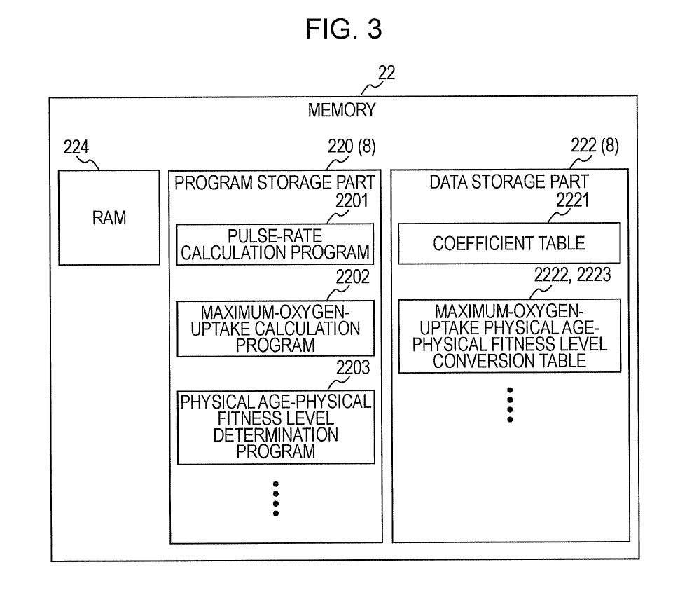

The first embodiment will now be described with reference to FIGS. 1 to 3. FIG. 1 is a block diagram illustrating an example of the functional configuration of a physical fitness determining apparatus according to the first embodiment. FIG. 2 is a block diagram illustrating an example of the hardware configuration of the physical fitness determining apparatus. FIG. 3 is a block diagram illustrating an example of the configuration of a memory in the physical fitness determining apparatus. The configurations and the processing shown in FIGS. 1 to 3 are only examples and the configurations and the processing of the physical fitness determining apparatus are not limited to the ones shown in FIGS. 1 to 3.

Referring to FIG. 1, a physical fitness determining apparatus 1 includes a physical-fitness determining unit 2, a pulse measuring unit 4, a display unit 6, and a data storage unit 8. The physical-fitness determining unit 2 calculates a maximum oxygen uptake by using a pulse rate that is measured in a calculation process and a conversion process described below to determine the physical age or the physical fitness level from the calculated maximum oxygen uptake by using the conversion means.

The physical-fitness determining unit 2 composes a computation function unit, a conversion function unit, and a determination function unit of the physical fitness determining apparatus 1. For example, the physical-fitness determining unit 2 includes a pulse-rate measurement processor 10, a maximum-pulse-rate calculation processor 12, a maximum-oxygen-uptake calculation processor 14, a physical age-physical fitness level determination processor 16, and a display processor 18.

The pulse-rate measurement processor 10 is an example of calculation means of a pulse rate. The pulse-rate measurement processor 10 calculates the pulse rate from information indicating a variation in the quantity of transmitted light acquired by the pulse measuring unit 4. The pulse-rate measurement processor 10 transmits the calculated pulse rate to the maximum-pulse-rate calculation processor 12, the data storage unit 8, and the like. If the quantity of the transmitted light is not varied or the pulse is not normally measured, the pulse-rate measurement processor 10 offers an advice or instruction indicating that the quantity of the transmitted light is not varied or the pulse is not normally measured.

The maximum-pulse-rate calculation processor 12 is an example of a computing unit. The maximum-pulse-rate calculation processor 12 calculates a maximum pulse rate (PRmax) corresponding to the age, etc. of a user and receives the pulse rate from the pulse-rate measurement processor 10 to calculate a pulse rate ratio (% PRmax) of the pulse rate relative to the maximum pulse rate. The maximum-pulse-rate calculation processor 12 transmits the calculated maximum pulse rate (PRmax) and pulse rate ratio (% PRmax) to the maximum-oxygen-uptake calculation processor 14. The maximum-pulse-rate calculation processor 12 may transmit the calculated maximum pulse rate (PRmax) to the data storage unit 8 to store the maximum pulse rate (PRmax) in the data storage unit 8 as, for example, personal information.

The maximum-oxygen-uptake calculation processor 14 is an example of the computing unit. The maximum-oxygen-uptake calculation processor 14 uses, for example, the measured pulse rate (PR), the maximum pulse rate (PRmax) and the pulse rate ratio (% PRmax) of the pulse rate relative to the maximum pulse rate, which are received from the maximum-pulse-rate calculation processor 12, and an oxygen uptake (Vo2) at rest to calculate a maximum oxygen uptake (Vo2max) in the calculation process and the conversion process described below. The maximum-oxygen-uptake calculation processor 14 transmits the maximum oxygen uptake (Vo2max) to the physical age-physical fitness level determination processor 16. As in the maximum-oxygen-uptake calculation processor 14, the maximum-oxygen-uptake calculation processor 14 may transmit the calculated maximum oxygen uptake (Vo2max) to the data storage unit 8 to store the maximum oxygen uptake (Vo2max) in the data storage unit 8 as, for example, the personal information.

The physical age-physical fitness level determination processor 16 is an example of a determining unit to determine the physical age or the physical fitness level of the user from the calculated maximum oxygen uptake (Vo2max). For example, a conversion table is used as the conversion means in which the maximum oxygen uptake (Vo2max) is associated with the physical fitness level or the physical age in this determination process. Specifically, the maximum oxygen uptake (Vo2max), which is the result of the calculation in the maximum-oxygen-uptake calculation processor 14, is used to determine the physical fitness level or the physical age from the conversion means. For example, computing means for computing the physical fitness level or the physical age from the maximum oxygen uptake may be used as the conversion means, instead of the conversion table.

The display processor 18 has a function of instructing the display unit 6 to perform display. For example, the display processor 18 receives information indicating the determination result from the physical age-physical fitness level determination processor 16 to create a display screen including the determination result and advice information corresponding to the determination result and transmits the display screen to the display unit 6. In addition, for example, the display processor 18 may instruct the user how to determine the physical age or the physical fitness level on the screen or may display guidance in the measurement of the pulse on the screen.

The pulse measuring unit 4 is an example of pulse measuring means for acquiring information about, for example, the pulse rate (PR) of the user. For example, the pulse measuring unit 4 measures the pulse rate from the blood flow state of a finger tip or an earlobe of the user. In the measurement of the pulse rate, the finger tip or the earlobe is irradiated with light to acquire the light transmitted through the finger tip or the earlobe.

The display unit 6 is an example of presentation means for presenting the determined physical age or physical fitness level to the user. The presentation means in the physical fitness determining apparatus 1 may perform, for example, audio presentation, instead of the presentation by the display unit 6.

The data storage unit 8 functions as a recording unit for recording, for example, the pulse rate, the maximum pulse rate, and the maximum oxygen uptake that are calculated. In addition, the data storage unit 8 is an example of a recording unit for recording, for example, programs for the calculation process and the conversion table used for the determination process.

The physical fitness determining apparatus 1 has the hardware configuration including, for example, a processor 20, a memory 22, the pulse measuring unit 4, the display unit 6, and an operation input unit 28, as shown in FIG. 2.

The processor 20 is, for example, a central processing unit (CPU). The processor 20 functions as computing means for executing an operating system (OS), various programs, etc. stored in the memory 22.

The memory 22 composes the data storage unit 8 described above, and functions as storing means for storing the calculation programs and recording means for recording data or the like. The memory 22 includes, for example, a program storage part 220, a data storage part 222, a random access memory (RAM) 224, as shown in FIG. 3. The program storage part 220 stores, for example, a pulse-rate calculation program 2201, a maximum-oxygen-uptake calculation program 2202, and a physical age-physical fitness level determination program 2203, in addition to the OS. The data storage part 222 records the acquired pulse information and the calculation results and stores, for example, a coefficient table 2221 and maximum-oxygen-uptake physical age-physical fitness level conversion tables 2222 and 2223, which are used in the conversion process described below.

The RAM 224 functions as a working area. The RAM 224 executes the various programs to compose, for example, the pulse-rate measurement processor 10, the maximum-pulse-rate calculation processor 12, the maximum-oxygen-uptake calculation processor 14, and the physical age-physical fitness level determination processor 16.

The pulse measuring unit 4 measures the pulse information from the blood flow state of a finger tip or an earlobe, as described above. The pulse measuring unit 4 is, for example, light receiving means for receiving the light transmitted through the finger tip, the earlobe, or the like. The display unit 6 composes the presentation means, as described above. The display unit 6 displays information indicating a health advice, in addition to the calculated physical age or physical fitness level. The display unit 6 is, for example, a liquid crystal display (LCD).

The operation input unit 28 is information input means. The operation input unit 28 includes a keypad unit including keys used for inputting symbols, such as letters, cursor keys used for selecting displayed information, and a determination key used for determining the selected information. In the measurement of the physical age or the physical fitness level, the operation input unit 28 is used to, for example, input age information about the user and input an instruction to start the measurement.

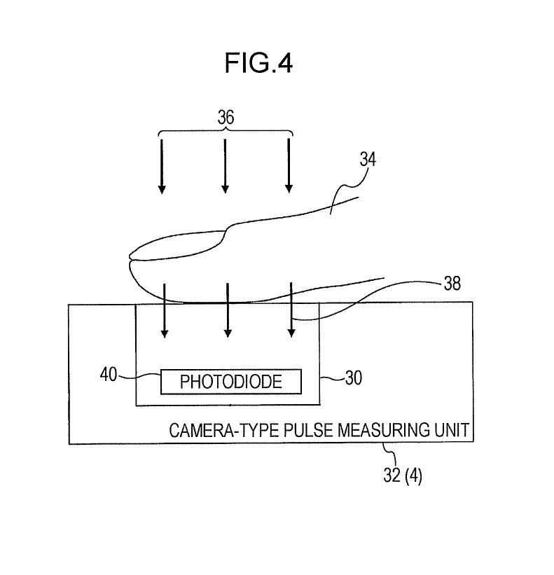

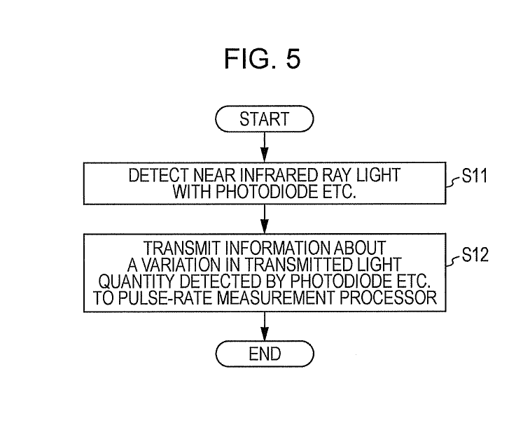

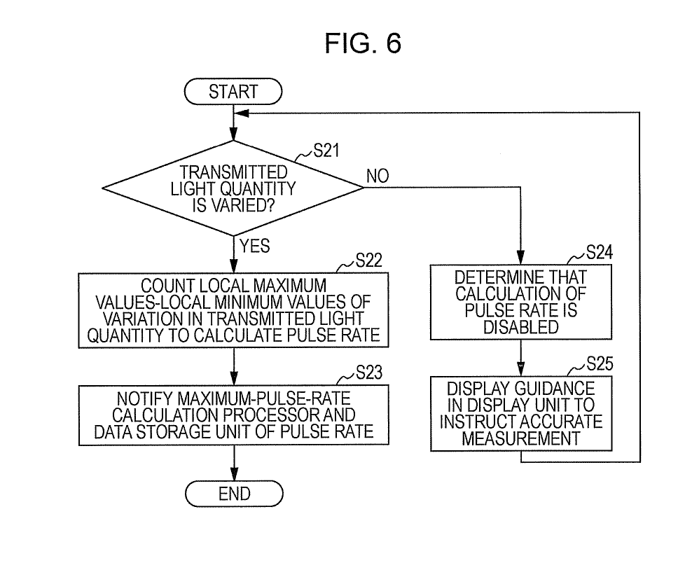

The measurement of the pulse in the pulse measuring unit 4 will now be described with reference to FIGS. 4 to 6. FIG. 4 illustrates an exemplary state of the measurement of the pulse by a camera-type pulse measuring unit. FIG. 5 is a flowchart illustrating an example of a process of measuring the pulse rate. FIG. 6 is a flowchart illustrating an example of a process of calculating the pulse rate. The configurations and the processing shown in FIGS. 4 to 6 are only examples and the configurations and the processing in the measurement of the pulse in the pulse measuring unit 4 are not limited to the ones shown in FIGS. 4 to 6.

The pulse measuring unit 4 is exemplified by a camera-type pulse measuring unit 32 using a camera 30 that acquires information about the pulse of a finger, as shown in FIG. 4. In the camera-type pulse measuring unit 32, the user places a finger 34 the pulse of which is to be measured on the camera 30 and the camera 30 captures light within the wavelength range of near infrared rays from transmitted light 38 that is incident on the finger 34 as incident (radiation) light 36, such as fluorescent light or sunlight, and that is transmitted through the finger 34. The near infrared rays in the components of the transmitted light 38 have the properties of being absorbed by the hemoglobin in blood. Since the bloods flow in the blood vessels is varied depending on the pulse, the quantity of transmitted light in the near infrared rays is reduced as the amount of blood flow increases. In contrast, the quantity of transmitted light is increased as the amount of blood flow decreases. The increase or decrease in the amount of blood flow is measured by, for example, detecting the quantity of transmitted light by a photodiode 40 in the camera 30 or detecting a variation in the luminance component on an image captured by the camera 30 to measure the pulse rate.

In the process of measuring the pulse rate in FIG. 5, in Step S11, near infrared ray light (transmitted light 38) that is incident as the incident light 36 and that is transmitted through the finger 34, which is a measurement target, is detected by, for example, the photodiode 40. In Step S12, information about a variation in the quantity of the transmitted light 38, detected by the photodiode 40, is transmitted to the pulse-rate measurement processor 10. The pulse-rate measurement processor 10 calculates the pulse rate on the basis of the information about the variation in the quantity of the transmitted light 38.

In the process of calculating the pulse rate in FIG. 6, in Step S21, it is determined whether the quantity of transmitted light is varied. If the quantity of transmitted light is not varied, the pulse rate may not be accurately measured. For example, the finger 34 may not be correctly placed on the camera 30.

If it is determined that the quantity of transmitted light is varied (YES in Step S21), that is, if the pulse rate can be measured, then in Step S22, the local maximum values and the local minimum values of the variation in the quantity of transmitted light are counted to calculate the pulse rate. In Step S23, the calculated pulse rate is notified to, for example, the maximum-pulse-rate calculation processor 12 and the data storage unit 8.

If the variation in the quantity of transmitted light cannot be detected (NO in Step S21), then in Step S24, it is determined that the calculation of the pulse rate is disabled. In this case, it may be determined that the user incorrectly places the finger 34 on the camera-type pulse measuring unit 32 or the finger 34 is not placed on the camera-type pulse measuring unit 32. In Step S25, the display processor 18 is notified that the variation in the quantity of transmitted light cannot be detected and guidance is displayed in, for example, the display unit 6 to instruct accurate measurement.

The guidance on how to place the finger 34 is not limited to the display in the display unit 6 and audio guidance may be offered by a speaker or the like. In addition, in the measurement of the pulse, the light incident on the finger is not limited to the infrared light emitted from a light source and the finger may be irradiated with, for example, sunlight. Furthermore, the pulse rate is not limitedly measured by the camera-type pulse measurement.

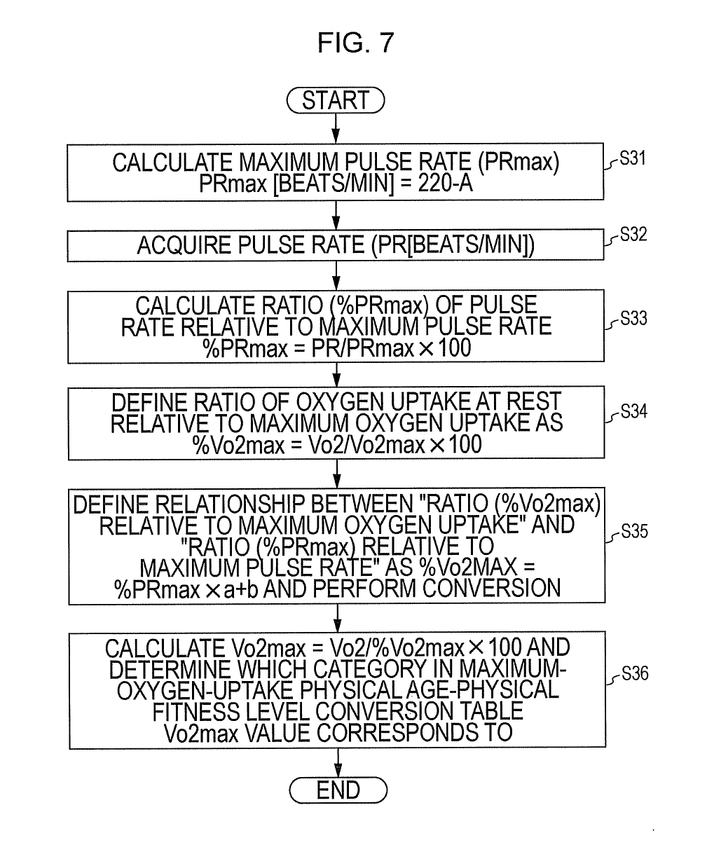

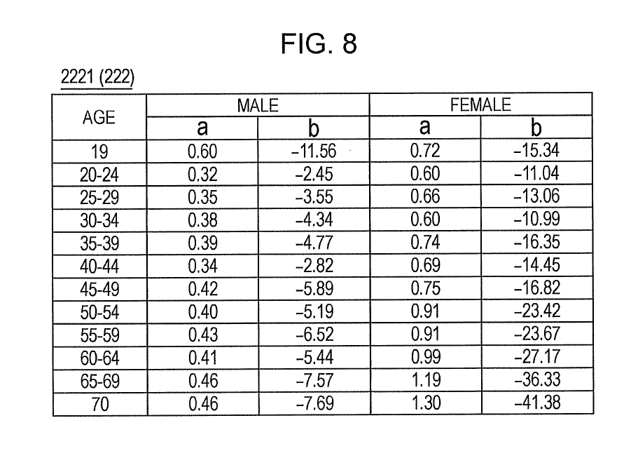

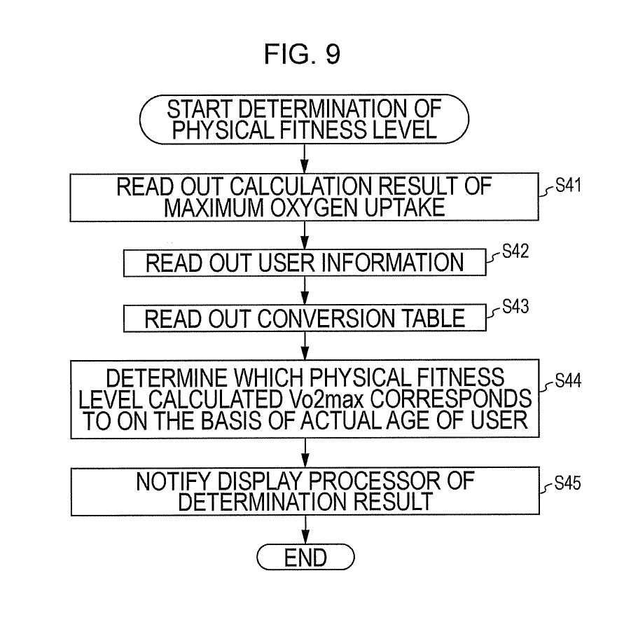

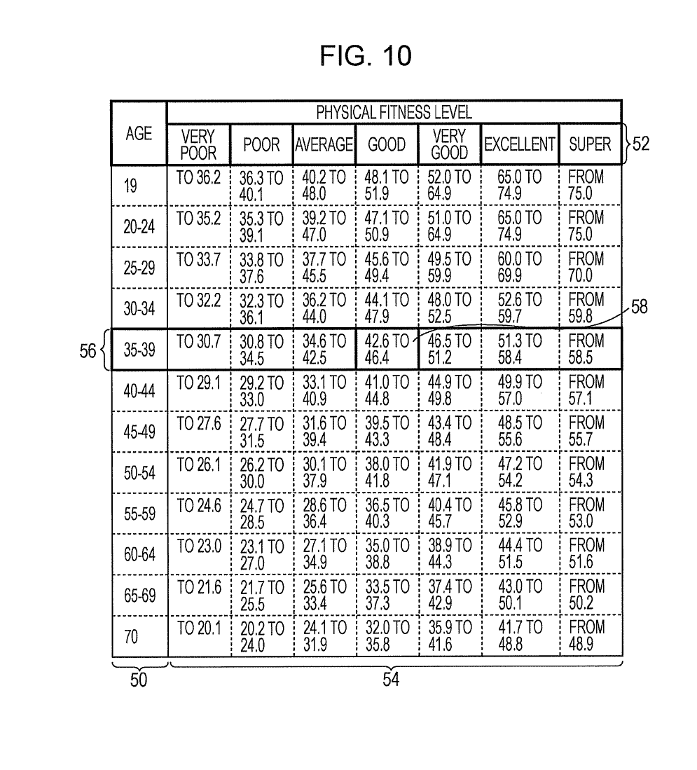

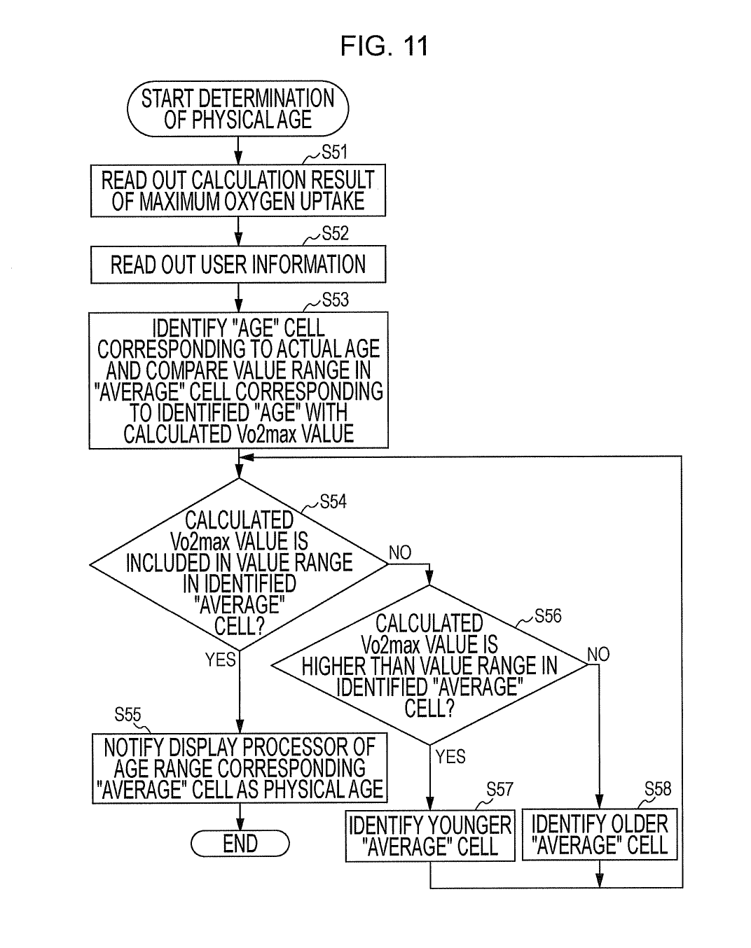

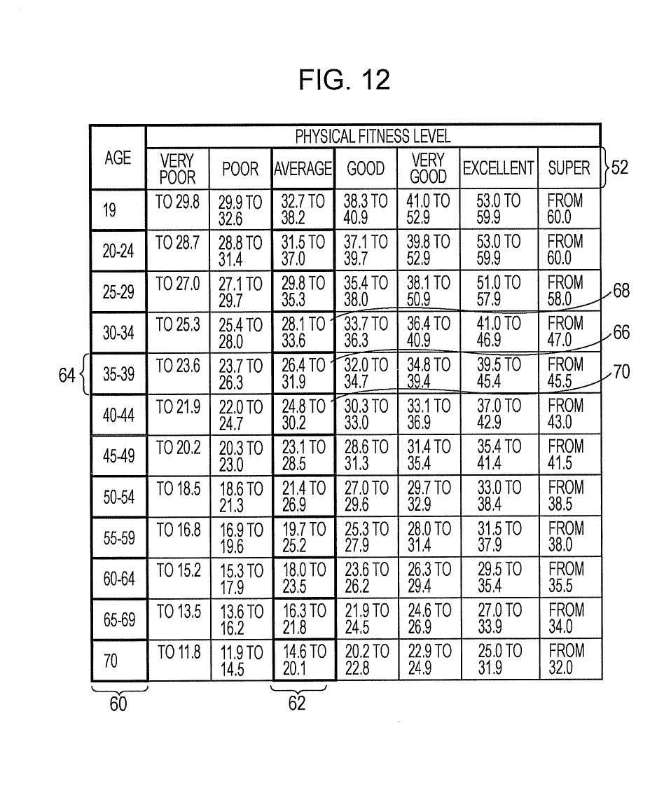

Determination of the physical fitness by the physical fitness determining method or the physical fitness determining program will now be described with reference to FIGS. 7 to 12. FIG. 7 is a flowchart illustrating an example of a process of determining physical fitness. FIG. 8 illustrates an example of a coefficient table. FIG. 9 is a flowchart illustrating an example of a process of determining a physical fitness level. FIG. 10 illustrates an example of how to determine the physical fitness level. FIG. 11 is a flowchart illustrating an example of a process of determining a physical age. FIG. 12 illustrates an example of how to determine the physical age. The processing, the steps, and the displayed values in FIGS. 7 to 12 are only examples, and the processing, the steps, and the displayed values in the determination of the physical fitness are not limited to the ones shown in FIGS. 7 to 12.

In the determination of the physical age or the physical fitness level, the maximum pulse rate, etc. are calculated by using the age of the user to calculate the pulse rate ratio of the pulse rate that is acquired relative to the maximum pulse rate. Next, the pulse rate ratio is converted into an oxygen uptake ratio, and the maximum oxygen uptake is calculated by using the oxygen uptake at rest. For example, calculation using a conversion equation is performed in the conversion between the pulse rate ratio and the oxygen uptake ratio. Then, the physical age or the physical fitness level is determined from the calculated value of the maximum oxygen uptake by using the conversion table described above.

Referring to FIG. 7, in Step S31, the maximum pulse rate (PRmax) is calculated. This calculation is performed in the maximum-pulse-rate calculation processor 12. The maximum pulse rate (PRmax) is a critical pulse rate indicating the pulse rate over which the user cannot do exercise and is calculated according to Equation (1):

PRmax[beats/min]=220−A (1)

In Equation (1), A denotes the actual age of the user to be measured. The actual age may be input by the user or data recorded in advance in, for example, the data storage unit 8 may be used as the actual age. The maximum pulse rate is reduced with the increasing age, as described in Equation (1). The calculated maximum pulse rate (PRmax) may be recorded in, for example, the data storage unit 8.

In Step S32, the pulse rate (PR [beats/min]) is acquired. As described above, the pulse rate is measured from, for example, the blood flow of a finger in the pulse measuring unit 4. In this measurement, the pulse rate during a predetermined time T is measured. For example, the pulse rate during one minute is measured.

In Step S33, the ratio of the current pulse rate relative to the maximum pulse rate, that is, the pulse rate ratio (% PRmax) of the acquired pulse rate relative to the maximum pulse rate is calculated by using the calculated maximum pulse rate (PRmax) and the measured pulse rate (PR). This calculation is expressed by Equation (2):

%PRmax=PR/PRmax×100 (2)

This calculation is performed in, for example, the maximum-pulse-rate calculation processor 12.

In Step S34, the ratio of the oxygen uptake (Vo2 [ml/kg/min]) at rest relative to the maximum oxygen uptake (Vo2max [ml/kg/min]), that is, the oxygen uptake ratio (% Vo2max) is defined. This definition is expressed by Equation (3):

%Vo2max=Vo2/Vo2max×100 (3)

This processing is performed in, for example, the maximum-oxygen-uptake calculation processor 14. For example, data stored in the data storage unit 8 may be read out to use the readout data in Equation (3). In Equation (3), a predetermined value X, for example, 3.5 [ml/kg/min] is used as the oxygen uptake (Vo2) at rest.

In Step S35, a correlation equation between the oxygen uptake ratio (% Vo2max) indicating the ratio of the oxygen uptake at rest relative to the maximum oxygen uptake and the pulse rate ratio (% PRmax) indicating the ratio of the current pulse rate relative to the maximum pulse rate is defined to perform conversion. Equation (4) is used as the correlation equation:

%Vo2max=%PRmax×a+b (4)

This correlation equation is means for converting the pulse rate ratio (% PRmax) calculated in Step S33 into the oxygen uptake ratio (% Vo2max). In Equation (4), a and b denote correlation coefficients between the pulse rate ratio and the oxygen uptake ratio. For example, in a linear equation on a graph in which the vertical axis represents the oxygen uptake ratio and the horizontal axis represents the pulse rate ratio, a denotes the gradient of the straight line and b denotes a segment intercepting the vertical axis. The coefficients a and b are determined from, for example, the coefficient table 2221 set for each gender and each age range, shown in FIG. 8. The coefficient table 2221 is stored in, for example, the data storage part 222. The definition process and the conversion process are performed in, for example, the maximum-oxygen-uptake calculation processor 14.

It is necessary to prepare information about the age and gender of the user to read out the coefficient data from the coefficient table 2221. The age information may be acquired in Step S31 and the gender information may be acquired from an operation by the user. Alternatively, the age information and the gender information may be registered in advance in the physical fitness determining apparatus 1.

Then, the maximum oxygen uptake (Vo2max) is calculated from the oxygen uptake (Vo2) at rest by using the oxygen uptake ratio (% Vo2max) resulting from the conversion. Equation (5) resulting from deformation of Equation (3) is used in the calculation of the maximum oxygen uptake.

In Step S36, the maximum oxygen uptake (Vo2max) is calculated from Equation (5) by using the oxygen uptake ratio (% Vo2max) resulting from the conversion in Step S35, and the physical age or the physical fitness level is determined on the basis of which category in the maximum-oxygen-uptake physical age-physical fitness level conversion table 2222 or 2223 (FIG. 10 or FIG. 12), which are conversion means, the calculated maximum oxygen uptake (Vo2max) corresponds to.

Vo2max=Vo2/%Vo2max×100 (5)

The determination of the physical age or the physical fitness level is not limited to the steps illustrated in the above flowcharts.

The determination of the physical age or the physical fitness level in Step S36 in FIG. 7 by the physical fitness determining method or the physical fitness determining program will now be specifically described. The determination processes described below are only examples and the determination of the physical age or the physical fitness level is not limited to the ones described below. In the process of determining the physical fitness level in FIG. 9, in Step S41, the physical age-physical fitness level determination processor 16 reads out the calculation result of the maximum oxygen uptake from, for example, the maximum-oxygen-uptake calculation processor 14 or the data storage unit 8. In Step S42, for example, age information and gender information used in the determination are read out as user information. The information acquired in the above calculation step may be used as the user information. In Step S43, the maximum-oxygen-uptake physical age-physical fitness level conversion table 2222 or 22223 corresponding to the gender information about the user is read out from, for example, the data storage unit 8.

In Step S44, the physical fitness level corresponding to the age information about the user and the calculated maximum oxygen uptake (Vo2max) is determined from the readout maximum-oxygen-uptake physical age-physical fitness level conversion table 2222 shown in FIG. 10. In the maximum-oxygen-uptake physical age-physical fitness level conversion table 2222, values 54 of the maximum oxygen uptake corresponding to determined physical fitness levels 52 are associated with age information 50 divided into age ranges. In the determination of the physical fitness level, for example, the physical fitness level allocated to the cell that includes the calculated value of the maximum oxygen uptake and that corresponds to the actual age of the user is determined.

As a specific example of the determination of the physical fitness level, a case in which the calculated value of the maximum oxygen uptake (Vo2max) of a 35-year male is equal to 43.0 will now be described with reference to FIG. 10.

In this case, a row 56 of an age range “35 to 39” surrounded by a solid line in the maximum-oxygen-uptake physical age-physical fitness level conversion table 2222 is referred to, as shown in FIG. 10. A cell including Vo2max=43.0 in the row 56 is referred to to determine the physical fitness level corresponding to this cell. In this case, since Vo2max=43.0 is included in a “GOOD” cell 58 in the row 56 of the age range “35 to 39”, the physical fitness level is determined to be “GOOD”.

In Step S45, the determination result is notified to the display processor 18 to cause the display unit 6 to display the determination result.

An example of the process of determining the physical age by the physical fitness determining method or the physical fitness determining program will now be described. The determination process described below is only an example and the determination of the physical age is not limited to the one described below.

Also in the process of determining the physical age in FIG. 11, in Step S51, the physical age-physical fitness level determination processor 16 reads out the calculation result of the maximum oxygen uptake from, for example, the maximum-oxygen-uptake calculation processor 14 or the data storage unit 8. In Step S52, for example, age information and gender information used in the determination are read out as the user information. Then, the maximum-oxygen-uptake physical age-physical fitness level conversion table 2222 or 2223 corresponding to the gender information about the user is read out.

Then, the maximum-oxygen-uptake physical age-physical fitness level conversion table 2222 or 2223 is used to move to the determination of the physical age. The determination of the physical age is based on an “AVERAGE” column indicating standard values of the maximum oxygen uptake for every age range. Specifically, the value of the maximum oxygen uptake (Vo2max) in the “AVERAGE” cell corresponding to the actual age is compared with the calculated value of the maximum oxygen uptake (Vo2max) to determine the physical age.

As a specific example of the determination of the physical age, a case in which the calculated value of the maximum oxygen uptake (Vo2max) of a 35-year female is equal to 33.0 will now be described with reference to FIG. 12.

First, an “AGE” column 60 and an “AVERAGE” column 62 including the values of the maximum oxygen uptake (Vo2max) are referred to. In Step S53, the value range in the “AVERAGE” cell corresponding to the actual age of the user is compared with the calculated value of the maximum oxygen uptake (Vo2max). Specifically, since the actual age of the user is 35, an “AVERAGE” cell 66 in an “AGE” row 64 corresponding to an age range “35 to 39” is identified. Then, the value range in the “AVERAGE” cell 66 is compared with the calculated value Vo2max=33.0.

In Step S54, it is determined whether the calculated value of the maximum oxygen uptake (Vo2max) is included in the value range in the identified “AVERAGE” cell. If it is determined that the calculated value of the maximum oxygen uptake (Vo2max) is included in the value range in the identified “AVERAGE” cell 66 (YES in Step S54), then in Step S55, the age range corresponding to the “AVERAGE” cell 66 is determined to be the physical age and the determination result is notified to the display processor 18.

If it is determined that the calculated value of the maximum oxygen uptake (Vo2max) is not included in the value range in the identified “AVERAGE” cell 66 (NO in Step S54), then in Step S56, it is determined whether the calculated value of the maximum oxygen uptake (Vo2max) is higher than the value range in the identified “AVERAGE” cell 66. The physical age is younger than the actual age if the calculated value of the maximum oxygen uptake (Vo2max) is higher than the value range in the “AVERAGE” cell 66 indicating the average maximum oxygen uptake corresponding to the actual age and, otherwise, the physical age is older than the actual age.

If the calculated value of the maximum oxygen uptake (Vo2max) is higher than the value range in the identified “AVERAGE” cell 66 (YES in Step S56), then in Step S57, an “AVERAGE” cell 68 that is younger than and is adjacent to the identified “AVERAGE” cell 66 is identified. If the calculated value of the maximum oxygen uptake (Vo2max) is lower than the value range in the identified “AVERAGE” cell 66 (NO in Step S56), then in Step S58, an “AVERAGE” cell 70 that is older than and is adjacent to the identified “AVERAGE” cell 66 is identified.

In the above specific example, the calculated value of the maximum oxygen uptake (Vo2max) is equal to 33.0 and the “AVERAGE” cell 66 corresponding to the age range “35 to 39” has a value range of “26.4 to 31.9”. Since the calculated value of the maximum oxygen uptake (Vo2max) is higher than the value range in the identified “AVERAGE” cell 66 (NO in Step S54 and YES in Step S56), the “AVERAGE” cell 68 corresponding to the younger age range “30 to 34” is identified as the determination target in Step S57.

After the new determination target is identified, the process goes back to the determination in Step S54 of whether the calculated value of the maximum oxygen uptake (Vo2max) is included in the value range in the “AVERAGE” cell 68 that is newly identified.

In the above specific example, the “AVERAGE” cell (the younger “AVERAGE” cell 68) corresponding to the newly identified age range “30 to 34” has a value range of “28.1 to 33.6”, which includes the calculated value, 33.0, of the maximum oxygen uptake (Vo2max) (YES in Step S54). Accordingly, the physical age of the user is determined to be in the age range “30 to 34” and the determination result is displayed in, for example, the display unit 6.

Although the physical fitness level of a male and the physical age of a female are determined in the above examples, the content of the determination is not determined by the gender. The physical age of a male and the physical fitness level of a female may be determined. The flowchart of the process of determining the physical age is separated from the flowchart of the process of determining the physical fitness level in the determination processes described above with reference to FIGS. 9 to 12 and the readout of the calculation results and the readout of the information about the user are separately performed in the respective processes, the determination of the physical age and the determination of the physical fitness level may be continuously performed. The processing order is not restricted in the processes of determining the physical fitness level and the physical age in FIGS. 9 and 11.

With the above configurations, since it is not necessary for the user to actually do exercise in order to measure a physiological variation in the body, such as the pulse rate, the physical age or the physical fitness level can be measured with no effect on, for example, the physical condition. In addition, since the physical age or the physical fitness level can be measured with a simple configuration and with a little amount of information to allow the user to easily confirm the health condition, a contribution to improvement in the health care awareness can be made. Furthermore, since it is not necessary to input physical feature data such as the body weight or the body height, in addition to the age information, as the personal information in the determination of the physical age or the physical fitness level, it is useful from the viewpoint of protection of the personal information.

The first embodiment of the present invention described above has the following features, advantages, and the like:

(1) According to the above first embodiment, since it is not necessary to use, for example, an electrocardiograph requiring a large-scale apparatus for the measurement, the physical age or the physical fitness level can be easily calculated with a simple configuration.

(2) According to the above first embodiment, the correlation equation between “the ratio (% Vo2max) relative to the maximum oxygen uptake” and the ratio (% PRmax) of the measured pulse rate relative to the maximum pulse rate “and the coefficient table for every age range and for every gender can be used to determine the physical age or the physical fitness level.

(3) According to the above first embodiment, the physical age or the physical fitness level can be calculated by using the maximum oxygen uptake and it is not necessary to prepare complicated information, such as the body weight or the power.

(4) According to the above first embodiment, it is not necessary to prepare information about, for example, the body weight or the power in the measurement of the maximum oxygen uptake and it is also not necessary to use a large-scale machine. In addition, since it is not necessary to input the personal information including the physical features, such as the body weight, which the user wants to keep secret, leakage of the personal information can be prevented even if a portable terminal apparatus in which the physical fitness determining apparatus or the physical fitness determining program are installed is lost.

### Second Embodiment

A second embodiment of the present invention concerns a notification process using the display unit.

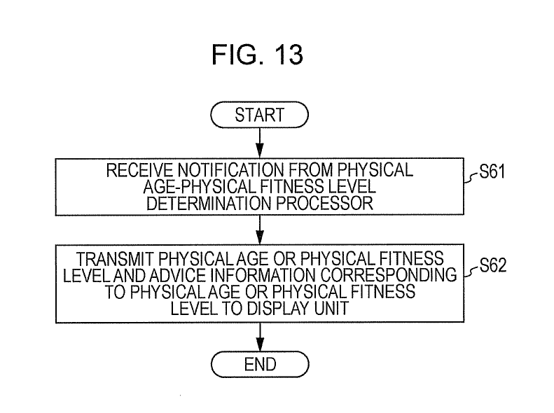

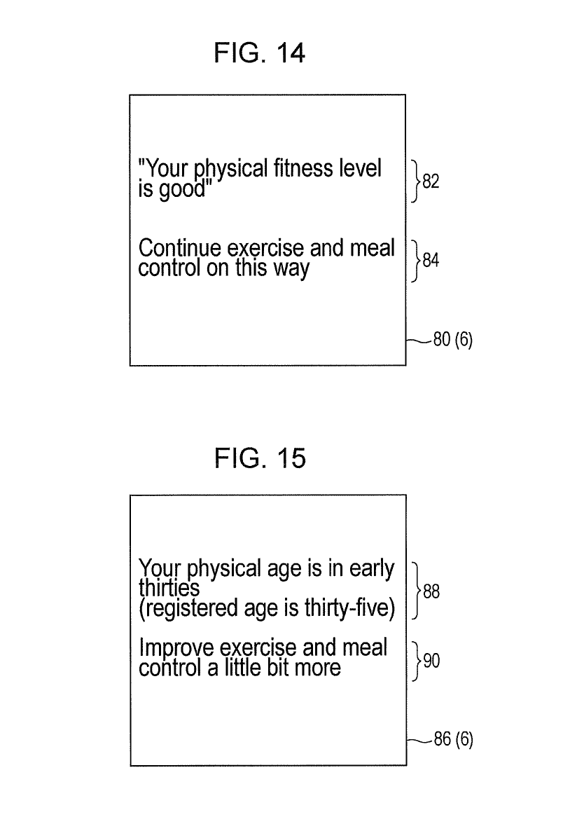

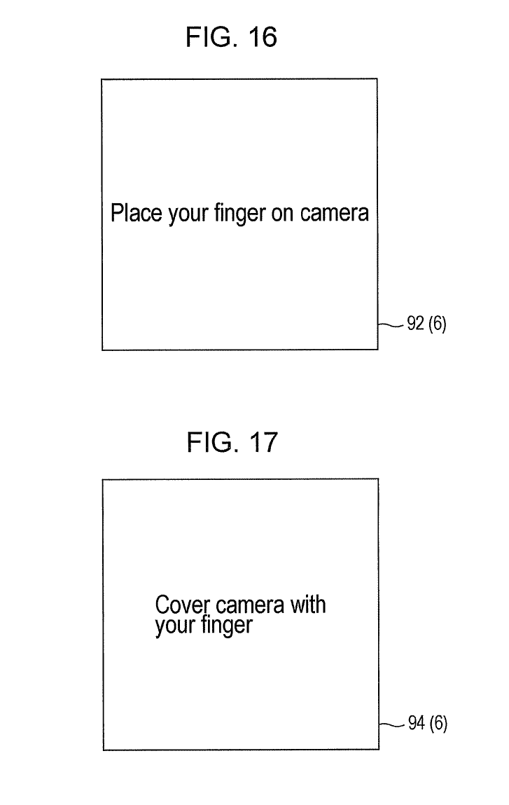

The second embodiment will now be described with reference to FIGS. 13 to 17. FIG. 13 is a flowchart illustrating an example of a process in the display processor according to the second embodiment. FIG. 14 illustrates an example of how to display the physical fitness level and an advice. FIG. 15 illustrates an example of how to display the physical age and an advice. FIG. 16 illustrates an example of displayed guidance in the measurement of the pulse. FIG. 17 illustrates an example of displayed guidance. The configurations, the processing, and the steps shown in FIGS. 13 to 17 are only examples and the configuration, the processing, and the steps in the notification process are not limited to the ones shown in FIGS. 13 to 17.

The display processor 18 in the physical fitness determining apparatus 1 presents the result of the determination of the physical age or the physical fitness level with, for example, the display unit 6 to notify the user of the determination result. In addition, the display processor 18 displays an advice concerning, for example, the health care for the user in the notification of the determination result. Furthermore, guidance concerning the determination of the physical fitness may be notified to the user in the notification process and, if the determination of the physical fitness cannot be normally performed, the failure in the determination may be notified.

In the process in the display processor in FIG. 13, in Step S61, the display processor 18 receives the result of determination of the physical age or the physical fitness level of the user from the physical age-physical fitness level determination processor 16. In Step S62, the display processor 18 creates, for example, a display screen 80 in FIG. 14 or a display screen 86 shown in FIG. 15, which is to be displayed in the display unit 6 and which includes information indicating the physical age or the physical fitness level of the user and a health care advice, and transmits the created display screen to the display unit 6.

In the display screen 80 for the physical fitness level in FIG. 14, for example, “Your physical fitness level is good” is displayed as a notification 82 of the physical fitness level. As described above, any of the determined physical fitness levels 52 in the maximum-oxygen-uptake physical age-physical fitness level conversion table 2222 or 2223 is displayed as the notification 82 of the physical fitness level. The display screen 80 also includes advice information 84 concerning the health care. For example, a table having the content of display corresponding to the determination result of the physical fitness level may be set for the advice information 84 and the table may be stored in the data storage unit 8. If the physical fitness level is “POOR” or “VERY POOR” in this case, a message to prompt the user to improve the exercise and/or the meal control may be displayed.

In the display screen 86 for the physical age in FIG. 15, for example, “Your physical age is in early thirties” is displayed as a notification 88 of the physical age. In this case, the actual age that is registered in, for example, the data storage unit 8 and that is used in the determination of the physical age may also be displayed for reference. The display screen 86 also includes advice information 90 concerning the health care. As in the display screen 80, a table having the content of display corresponding to the determination result of the physical age may be set for the advice information 90 and the table may be stored in the data storage unit 8 for display.

The physical age, the physical fitness level, and the advice may be displayed together on one screen.

FIGS. 16 and 17 illustrates examples of the guidance displayed in the measurement of the pulse rate in the notification with the display unit 6.

The examples of the displayed guidance in FIGS. 16 and 17 are examples of guidance in the camera-type pulse measuring unit 32 described above. Specifically, a guidance display screen 92 to prompt the user to place the finger 34 on the camera 30 is displayed in the display unit 6 in response to start of the determination of the physical age or the physical fitness level.

In the process of calculating the pulse rate in FIG. 6, for example, a guidance display screen 94 to prompt the user to cover the camera 30 with the finger 34 is displayed (Step S25 in FIG. 6) if the pulse cannot be correctly measured (NO in Step S21 in FIG. 6). Alternatively, for example, a guidance display screen to prompt the user to change the position of the finger 34 may be displayed.

The notification of the physical age, the physical fitness level, the advice corresponding to the physical age or the physical fitness level, and the guidance in, for example, the calculation of the pulse rate are not limited to the notification with the display unit 6. For example, audio notification may be performed.

With the above configurations, it is possible to improve the health care awareness of the user by notifying the user of the physical age or the physical fitness level determined in the physical fitness determining apparatus 1 and offering the advice based on the calculation result. In addition, displaying the guidance to prompt the user to accurately measure the pulse in the calculation of the pulse rate allows the pulse to be accurately measured to improve the usefulness.

### Third Embodiment

A third embodiment of the present invention concerns an example of a portable terminal apparatus realizing the physical fitness determining apparatus.

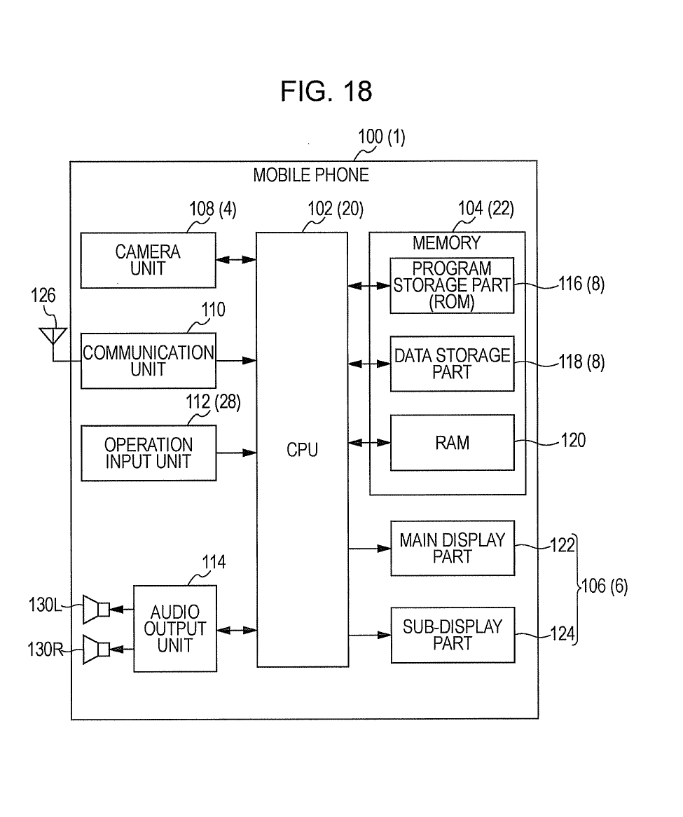

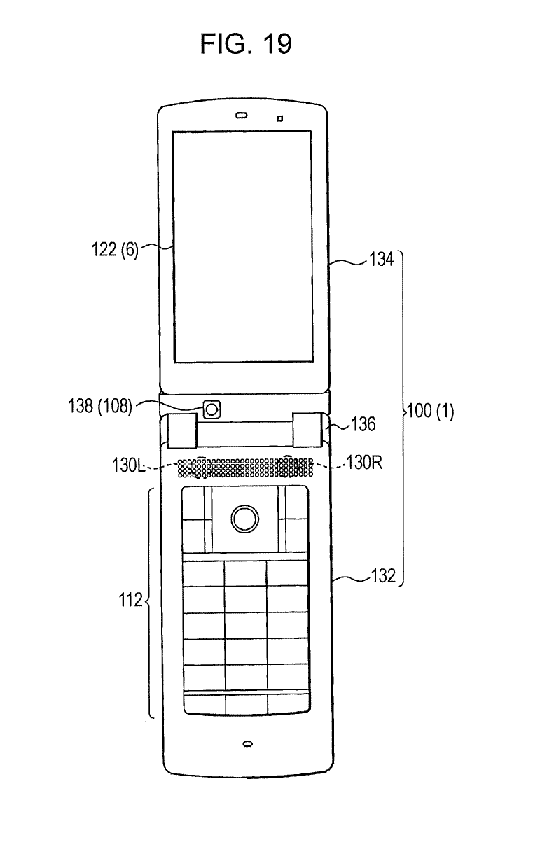

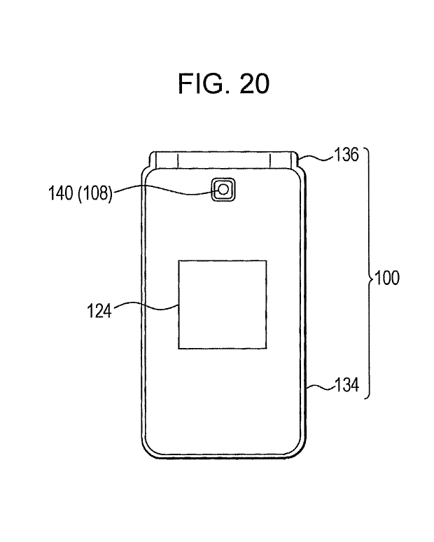

The third embodiment will now be described with reference to FIGS. 18 to 20. FIG. 18 is a block diagram illustrating an example of the hardware configuration of a mobile phone according to the third embodiment. FIG. 19 illustrates an example of the external structure of the mobile phone in an open state. FIG. 20 illustrates an example of the external structure of the mobile phone in a closed state. The configuration and the structures shown in FIGS. 18 to 20 are only examples and the configuration and the structures of the mobile phone are not limited to the ones shown in FIGS. 18 to 20.

A mobile phone 100 is an example of the physical fitness determining apparatus, the physical fitness determining method, the physical fitness determining program, or the portable terminal apparatus. For example, the mobile phone 100 includes a CPU 102, a memory 104, a display unit 106, a camera unit 108, a communication unit 110, an operation input unit 112, and an audio output unit 114.

The CPU 102 corresponds to the processor 20 in the physical fitness determining apparatus 1 and is an example of arithmetic processing means, such as a program executed by the mobile phone 100. The CPU 102 executes, for example, the OS, the pulse-rate calculation program, and the maximum-oxygen-uptake calculation program.

The memory 104 corresponds to the memory 22. For example, the memory 104 includes a program storage part 116, a data storage part 118, and a RAM 120. The program storage part 116 is, for example, a read only memory (ROM) storing the OS of the mobile phone 100, the physical age-physical fitness level determination program, and other programs. The data storage part 118 composes the data storage unit 8 described above. For example, the data storage part 118 stores information about the pulse rate that is measured and the maximum oxygen uptake that is calculated and also stores the coefficient table 2221 and the maximum-oxygen-uptake physical age-physical fitness level conversion table 2222 or 2223.

The RAM 120 functions as a working area in which, for example, the calculation process is performed. The RAM 120 executes the above programs stored in the program storage part 116 to compose, for example, the pulse-rate measurement processor 10, the maximum-pulse-rate calculation processor 12, the maximum-oxygen-uptake calculation processor 14, the physical age-physical fitness level determination processor 16, and the display processor 18.

The display unit 106 composes notification means for notifying the physical age or the physical fitness level that is calculated and a health advice based on the physical age or the physical fitness level. The display unit 106 includes a main display part 122 and a sub-display part 124, which are, for example, liquid crystal displays (LCDs).

The camera unit 108 is an example of image pickup means for capturing an image of a finger tip or a hand in the measurement of the pulse rate. The camera unit 108 is, for example, a digital camera and composes the memory 104 that reads out the blood flow state of a finger tip or the like.

The communication unit 110 includes an antenna 126. The communication unit 110 controls communication with an external base station, another portable terminal apparatus, or a server and data exchange by wireless communication over, for example, a telephone line or a network. In the determination of the physical age or the physical fitness level, for example, the calculation program described above may be downloaded from an external server or the like through the communication unit 110, if necessary.

The operation input unit 112 is information input means. The operation input unit 112 includes a keypad unit including keys used for inputting symbols, such as letters, cursor keys used for selecting displayed information, and a determination key used for determining the selected information. The audio output unit 114 includes, for example, speakers 130R and 130L and reproduces an audio signal as a sound or voice.

For example, a soft key function in which icons displayed on the display screen of the display unit 106 are associated with the input keys may be used as the operation input unit 112. Alternatively, the operation input unit 112 may be a so-called touch panel that is integrated with the display unit 106 and that detects the position where a finger of the user touches on an operation command displayed on the display screen to recognize the position on the screen and issues an instruction to the computer.

Referring to FIG. 19, the mobile phone 100 includes a first casing 132 and a second casing 134. A hinge part 136 connects the first casing 132 and the second casing 134 and allows them to be folded. The first casing 132 includes the operation input unit 112 and the speakers 130L and 130R. The operation input unit 112 includes, for example, the keypad unit. The second casing 134 includes the main display part 122 and an in-camera 138.

Referring to FIG. 20, the mobile phone 100 includes the sub-display part 124 at a central part on an external face of the second casing 134 and includes an out-camera 140 toward the hinge part 136.

With the above configurations, the physical fitness can be determined with a simple configuration by using the mobile phone 100 which the user daily carries and uses and the determination result can be notified to the user. Accordingly, it is possible to present information that is useful for improving the health care awareness, thus achieving advantages similar to the ones of the first embodiment also in the third embodiment.

### Fourth Embodiment

A fourth embodiment of the present invention concerns an example of a pulse measuring unit using an ear clip.

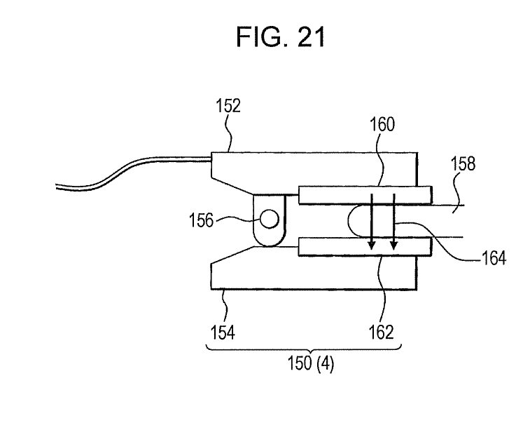

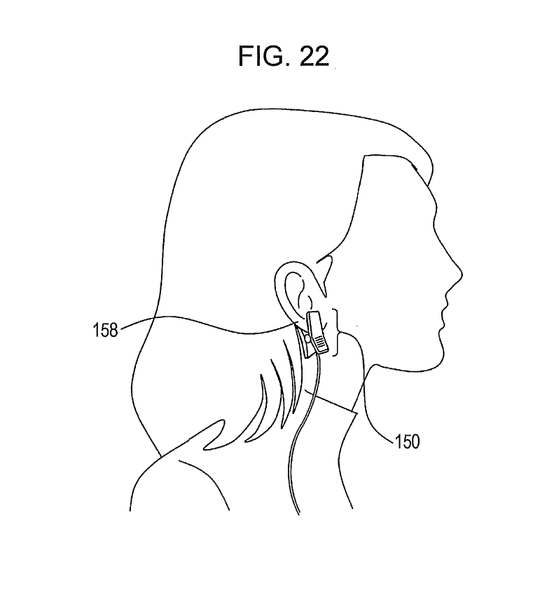

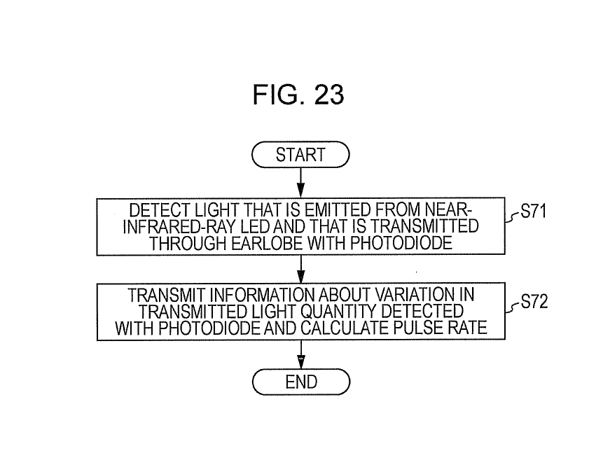

The fourth embodiment will now be described with reference to FIGS. 21 to 23. FIG. 21 illustrates an example of the structure of an ear-clip-type pulse measuring unit according to the fourth embodiment. FIG. 22 illustrates an example of how to wear the ear-clip-type pulse measuring unit. FIG. 23 is a flowchart illustrating an example of a process of calculating the pulse rate in the ear-clip-type pulse measuring unit. The configuration and the processing shown in FIGS. 21 to 23 are only examples and the configuration and the processing of the ear-clip-type pulse measuring unit are not limited to the ones shown in FIGS. 21 to 23.

A ear-clip-type pulse measuring unit 150 according to the fourth embodiment is an example of the pulse measuring unit 4. Referring to FIG. 21, the ear-clip-type pulse measuring unit 150 includes two casing units 152 and 154 and a hinge part 156 that connects the casing unit 152 and the casing unit 154. The ear-clip-type pulse measuring unit 150 is configured in a clip shape in which an earlobe 158 the pulse of which is to be measured is clipped between the casing unit 152 and the casing unit 154. A near-infrared-ray LED 160 serving as a light source is provided on a surface of the casing unit 152, which opposes the casing unit 154. A photodiode 162 serving as light receiving means is provided on a surface of the casing unit 154, which opposes the casing unit 152.

When the ear-clip-type pulse measuring unit 150 is used, the earlobe 158 of the user is clipped between the casing units 152 and 154, as shown in FIG. 22. Near infrared ray light emitted from the near-infrared-ray LED 160 is transmitted through the earlobe 158 and transmitted light 164 is detected with the photodiode 162. The ear-clip-type pulse measuring unit 150 measures the pulse from a variation in the amount of blood flow based on the quantity of transmitted light, as in the pulse measurement using a camera, described above.

In the process of calculating the pulse rate by the ear-clip-type pulse measuring unit 150 shown in FIG. 23, in Step S71, the light (transmitted light 164) that is emitted from the near-infrared-ray LED 160 and that is transmitted through the earlobe 158 is detected with the photodiode 162. In Step S72, information about the variation in the quantity of the detected transmitted light 164 is transmitted to the pulse-rate measurement processor 10. The pulse-rate measurement processor 10 calculates the pulse rate on the basis of the information about the variation.

The target of the measurement of the pulse rate with a clip is not limited to the blood flow of the earlobe 158. For example, a finger tip of the user is clipped to measure the pulse.

Also with the above configuration, it is possible to measure the pulse rate with a simple configuration and to use the measured pulse rate for the determination of the physical fitness, thus achieving advantages similar to the ones in the above embodiments.

### Other Embodiments

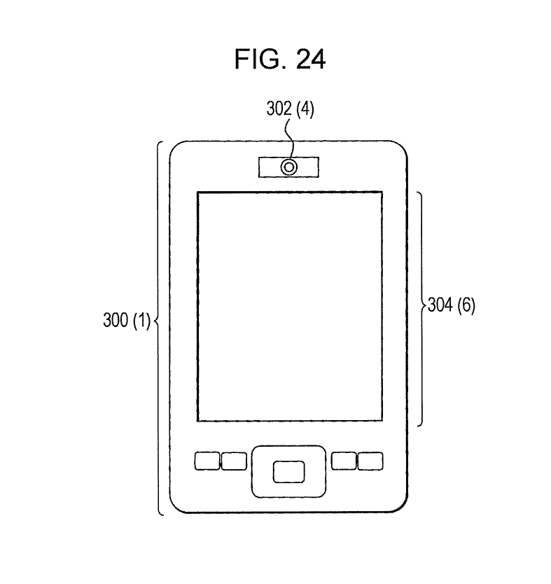

(1) Although the physical fitness determining apparatus, the physical fitness determining method, the physical fitness determining program, or the portable terminal apparatus is exemplified by the mobile phone 100 in the above embodiments, the physical fitness determining apparatus, the physical fitness determining method, the physical fitness determining program, or the portable terminal apparatus is not limited to the mobile phone 100. For example, as shown in FIG. 24, a personal digital assistant (PDA) 300 may be used. The PDA 300 includes, for example, a camera 302 composing the pulse measuring unit 4 and a display unit 304 composing the presentation means.

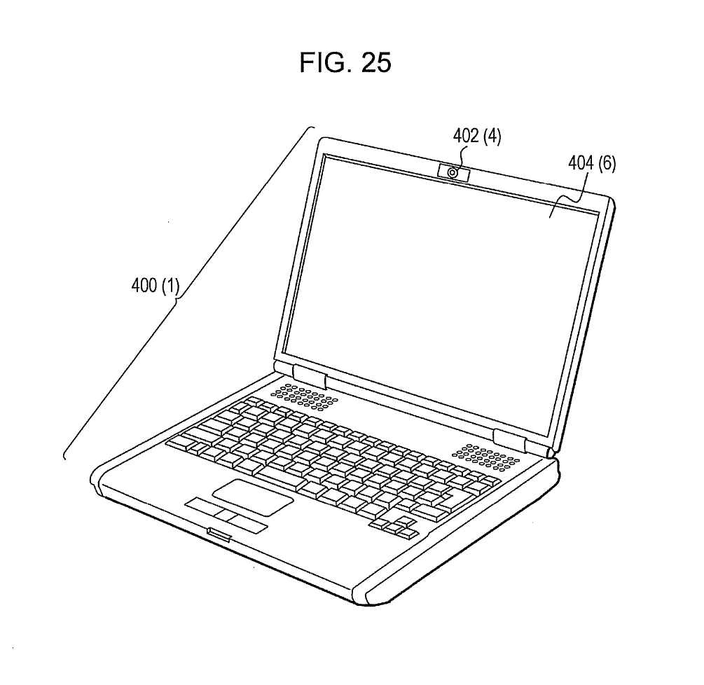

(2) In addition, as shown in FIG. 25, a personal computer (PC) 400 may be used. The PC 400 includes, for example, a camera 402 composing the pulse measuring unit 4 and a display unit 404 composing the presentation means for presenting the determination result. Also with the above configurations, objects similar to the ones in the above embodiments can be attained.

(3) Although the adjacent younger or older cell (68 or 70) is referred to if the calculated maximum oxygen uptake (Vo2max) is not included in the value range in the “AVERAGE” cell 66 corresponding to the actual age, as shown in FIG. 12, in the determination of the physical fitness in the above embodiments, the determination of the physical fitness is not limited to the above one. For example, if the calculated maximum oxygen uptake (Vo2max) is included in multiple physical age ranges in the “AVERAGE” column 62, any of the age ranges may be determined to the physical age. Specifically, Vo2max=33.0 is included in all of the cells younger than the age range “30 to 34” in the “AVERAGE” column 62 in the above example. Accordingly, the age range younger than the age range “30 to 34” may be determined to be the physical age in the determination of the physical age.

(4) If the calculated maximum oxygen uptake (Vo2max) is included in multiple physical age ranges in the determination of the physical age, the determination target may be varied depending on, for example, the number of times of calculation of the physical age. Specifically, the number of times of calculation may be counted as the calculation record of the physical age and, if the maximum oxygen uptake (Vo2max) included in multiple physical age ranges is calculated multiple times, the target cell of the determination of the physical age may be changed to the younger or older cell. Also with this configuration, it is possible to achieve advantages similar to the ones in the above embodiments.

(5) Although the amount of blood flow of a finger tip or an earlobe is detected on the basis of the quantity of transmitted light in the measurement of the pulse rate in the above embodiments, the measurement of the pulse rate is not limited to the above one. For example, the pulse may be measured with pressure applied to a wrist or the heart beat of the user may be measured with an audio sensor.

A physical fitness determining apparatus determining physical fitness by using an oxygen uptake includes a maximum-oxygen-uptake computing unit that calculates a pulse rate ratio of a pulse rate that is acquired relative to a maximum pulse rate, converts the pulse rate ratio into an oxygen uptake ratio, and calculates a maximum oxygen uptake from an oxygen uptake at rest by using the oxygen uptake ratio; conversion means in which the maximum oxygen uptake is associated with a physical fitness level; and a determining unit that determines the physical fitness level from the conversion means by using the maximum oxygen uptake calculated in the maximum-oxygen-uptake computing unit.

The maximum oxygen uptake is associated with an age in the conversion means. The determining unit determines the age from the maximum oxygen uptake in the conversion means by using the maximum oxygen uptake calculated in the maximum-oxygen-uptake computing unit to determine the age to be a physical age.

The physical fitness determining apparatus further includes pulse-rate measuring means for measuring the pulse rate.

The conversion means is a conversion table or computing means in which the maximum oxygen uptake is associated with the physical fitness level for every age and the physical fitness level or the physical age is determined from the maximum oxygen uptake.

The physical fitness determining apparatus further includes presentation means for presenting the physical fitness level.

The physical fitness determining apparatus further includes presentation means for presenting the physical age.

The maximum oxygen uptake associated with a standard physical fitness level is used in the determination of the age.

The physical fitness determining apparatus, the physical fitness determining method, the physical fitness determining program, and the portable terminal apparatus according to the embodiments of the present invention are useful because they can determine and notify the physical fitness with a simple configuration by using information, such as a pulse or an oxygen uptake, which a user can easily acquire to attain the health promotion of the user.

All examples and conditional language recited herein are intended for pedagogical purposes to aid the reader in understanding the invention and the concepts contributed by the inventor to furthering the art, and are to be construed as being without limitation to such specifically recited examples and condition, nor does the organization of such examples in the specification relate to a showing of superiority and inferiority of the invention. Although the embodiment of the present inventions have been described in detail, it should be understood that the various changes, substitutions, and alternations could be made hereto without departing from the spirit and scope of the invention.

What is claimed is:

1. An apparatus for determining physical fitness levels, comprising:
a pulse measuring unit which measures a pulse rate of a user at rest;
a measurement processor which acquires a pulse rate ratio of the pulse rate relative to a maximum pulse rate which is determined by an actual age of the user, estimates an oxygen uptake ratio on the basis of the pulse rate ratio and the actual age, and calculates a maximum oxygen uptake from an oxygen uptake at rest by using the oxygen uptake ratio;
a conversion table which associates a maximum oxygen uptake with a physical fitness level; and
a determiner which determines the physical fitness level in accordance with the calculated maximum oxygen uptake and the conversion table.

2. The apparatus according to claim 1, wherein the conversion table associates a maximum oxygen uptake with an age, and the determiner acquires the age by using the calculated maximum oxygen uptake and determines the age as a physical age.

3. The apparatus according to claim 1, wherein the conversion table associates a maximum oxygen uptake with a physical fitness level with respect to each age, and the determiner determines a physical fitness level or a physical age in accordance with the conversion table.

4. The apparatus according to claim 1, further comprising:
a display which displays the physical fitness level.

5. The apparatus according to claim 1, further comprising:
a display which displays the physical age.

6. The apparatus according to claim 2, wherein the determiner determines the physical age by using a maximum oxygen uptake corresponding to an average of the physical fitness level.

7. A method for determining physical fitness levels, comprising:
measuring a pulse rate of a user at rest;
acquiring a pulse rate ratio of the pulse rate relative to a maximum pulse rate determined by an actual age of the user;
estimating an oxygen uptake ratio on the basis of the pulse rate ratio and the actual age;
calculating a maximum oxygen uptake from an oxygen uptake at rest by using the oxygen uptake ratio; and
determining a physical fitness level in accordance with the calculated maximum oxygen uptake and a conversion table associating a maximum oxygen uptake with a physical fitness level.

8. A mobile terminal apparatus having a function for determining physical fitness levels, comprising:
a pulse measuring unit which measures a pulse rate of a user at rest;
a measurement processor which acquires a pulse rate ratio of the pulse rate relative to a maximum pulse rate determined by an actual age of the user, estimates an oxygen uptake ratio on the basis of the pulse rate ratio and the actual age, and calculates a maximum oxygen uptake from an oxygen uptake at rest by using the oxygen uptake ratio;
a conversion table which associates a maximum oxygen uptake with a physical fitness level; and
a determiner which determines the physical fitness level in accordance with the calculated maximum oxygen uptake and the conversion table.
